SEQUENCE LISTING (2 sequences)

SEQ ID NO: 1 - protein, 489 aa, Homo sapiens
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 2 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

(19) United States
(12) Patent Application Publication
(10) Pub. No.: US 2006/0057105 A1
(43) Pub. Date: Mar. 16, 2006

(54) Methods of treating ocular inflammation and allergy

(76) Inventors: Michael E. Stern, Mission Viejo, CA (US); Jerry Y. Niederkorn, Coppell, TX (US); Karen F. Siemasko, Irvine, CA (US)

Correspondence Address: JOEL B. GERMAN;ALLERGAN, INC., 2525 Dupont Drive, T2-7H, Irvine, CA, 92612, US

(21) Appl. No.: 11/210,251

(22) Filed: Aug. 22, 2005

Related U.S. Application Data
(60) Provisional application No. 60/603,301, filed on Aug. 20, 2004.

(51) Int. Cl. A61K 48/00; A61K 38/21; A61K 39/395; A61K 38/17

(52) U.S. Cl. 424/85.4; 514/12; 514/44; 424/145.1

(57) ABSTRACT

The present specification discloses methods of treating ocular inflammation and ocular allergy through the administration of interferon inhibitors to a mammal, including a human, in need thereof.

This patent application claims benefit of priority under 35 USC § 119(e) to provisional patent application 60/603,301, filed Aug. 20, 2004, which is hereby incorporated by reference herein.

### BACKGROUND OF THE INVENTION

Conventionally, immune responses have been divided into two types: humoral immunity, mediated by antibodies secreted by B lymphocytes, and cellular immunity, mediated by T lymphocytes. In actuality, most immune responses involve both B and T lymphocytes, and the activation of T lymphocytes requires the participation of a third type of cell, known as an antigen-presenting cell or APC.

T lymphocytes respond to an immunological stimulus by secreting a variety of cytokines. T lymphocytes may display either the CD4 accessory molecule or the CD8 accessory molecule on their surface. Among CD4+ T helper lymphocytes two cell types have been characterized based upon the array of cytokines they secrete. Th1 cells produce lymphokines including interleukin 2 (IL-2) and gamma interferon (IFN-γ), but do not produce IL-4, IL-5, IL-6, IL-10, and IL-13. Th2 lymphocytes produce IL-4, IL-5, IL-6, IL-10, and IL-13, but do not produce IL-2 and IFN-γ. Some clones of CD8+ T lymphocytes and non-Th1 CD4+ T lymphocytes have also been shown to secrete IFN-γ.

According to the prevailing paradigm, Th1 cells are associated with cell-mediated autoimmune disease and Th2 cells regulate humoral mediated diseases such as lupus and allergy. Th1 cells have been shown to protect against intracellular infection, activate phagocytes, induce IgG₂ₐ antibodies and promote delayed-type hypersensitivity responses. Th2 cells have been shown to protect against extracellular injection, activate eosinophils, induce IgE-mediated allergic reactions, and promote IgG1 associated humoral responses.

The pathogenesis of lupis, once thought to be strictly mediated by Th2 T cells, has recently been shown to involve the participation of IFN-γ, which are not expressed by these cells. Mouse models such as the MRL-Fas^{lpr} strain which are predisposed to develop lupus show overexpression of IFN-γ, and normal (non-predisposed) transgenic mice expressing high levels of IFN-γ developed a T-cell dependent lupus like syndrome. Theofilopoulos, A. N., et al., 3 ARTHRITIS RES. 136-141 (2001). Mice treated with anti-IFN-γ antibody were had significantly delayed onset of lupus than untreated mice. Also, glomerulonephritis and early death were prevented in mice heterozygous for the deletion of the IFN-γ gene (that is, having about 50% of a reduction in IFN-γ levels). Intramuscular injection of a plasmid encoding a fusion protein comprising the IFN-γ receptor (IFN-γR) and an IgG₁Fc fragment in MRL-Fas^{lpr} mice resulted in a concomitant reduction in IFN-γ serum levels and all disease parameters. Id. Other studies using this plasmid have found that it effectively reduces many symptoms of autoimmune diabetes in diabetic mice. Prud'homme et al., 6 GENE THERAPY 771-777 (1999).

IFN-γ has recently been shown to play a role in the pathophysiology of Th2 inflammation in a mouse model on allergic conjunctivitis. IFN-γ was required in order for mice to mount a significant neutrophil or eosinophil response to ragweed sensitization and challenge. Stem, M. E. et al., Presentation: 23^{rd} Biennial Cornea Research Conference, Boston, Mass. (Oct. 3, 2003).

All references cites in this application are hereby incorporated by reference herein.

### SUMMARY OF THE INVENTION

The present invention is directed to methods for the treatment of ocular inflammation and allergy in humans comprising contacting the ocular surface with an IFN-γ inhibitor. The IFN-γ inhibitor comprises any agent able to prevent IFN-γ mediated signal transduction by the IFN-γR complex, and may include, without limitation, an anti-IFN-γ antibody such as a monoclonal anti-IFN-γ antibody, a soluble IFN-γR fragment able to bind IFN-γ, and a small molecule antagonist of the IFN-γR. By “small molecule” is meant a molecule other than a polypeptide or nucleic acid.

Techniques for the generation of antigen specific monoclonal antibodies are now well known in the art. One may use a modification of one such technique, that employed by Kohler and Milstein (Kohler, G. and Milstein, C., Nature, 256: 495497 (1975); as follows:

Human melanoma cells that have been treated with 8-azaguanine for 48 hours are removed from the drug and grown to a maximum concentration of 500,000 cells per ml. Mice are previously immunized with human IFN-γ, and then boosted IV 72 hours prior to hybridization. Growth media is Dulbecco's modified Eagle's medium (DMEM) supplemented with sodium bicarbonate plus non-essential amino acids, penicillin-streptomycin, L-glutamine and hypoxanthine plus thymidine (HT). For the serum-containing media (used for the final plating), add 5-10% fetal calf serum. 40% PEG is prepared using serum-free medium (SFM) as the solvent. The stock is stored at −30° C.

All cells and solutions are maintained at room temperature or 37° C.

Day 0

A) Preparation of Tumor Cells

1. Check water bath for cleanliness. Correct water volume and temperature (equilibrated to 37° C. with the lid off).
2. Soak the spleen crusher in 70% ethanol for 5-10 minutes and let it dry sterilely in the hood.
3. Count the tumor cells. Begin washing the tumor cells with SFM as follows:
4. Centrifuge the cells out of serum containing media in 50 ml conical tubes (1200 RPM for 5 minutes). Discard the supernatant.
5. Carefully resuspend the cell pellet in approximately 1 ml of SFM. Transfer the cells to a new 50 ml conical tube.
6. Repeat the last two steps, transferring all of the tumor cells into a new tube.
7. Add 30-50 ml of SFM to the cell suspension and centrifuge again. Discard the supernatant.
8. Repeat the last step two more times.
   B) Preparation of Spleen Cells

While washing the tumor cells, prepare the spleen cells as follows:
1. Bleed the animal for antisera and let the blood clot at room temperature for 1-2 hours. Transfer the blood overnight to a 4° C. refrigerator before removing the clot.
2. Remove the spleen and place it in a sterile Petri dish containing 10 ml of SFM. Move this Petri dish into the hood.
3. Transfer the spleen with sterile forceps into new sterile Petri dish containing 10 ml SFM. This step reduces the nonsterile contaminants that may be present in the first Petri dish).
4. Crush the spleen with the sterile spleen crusher.
5. Carefully pipet the spleen cell mixture up and down in the Petri dish to break up large cell clumps.
6. Transfer the cell suspension to a 15 ml conical tube.
7. Let debris settle out of the cell solution (24 minutes).
8. Transfer clean supernatant into a new 15 ml conical tube.
9. Centrifuge the cells for 5 minutes at 1200 RPM.
10. Resuspend the cell pellet in 10 ml of SFM and count 5 μl on a hemocytometer.
   C) Cell Fusion

The ideal cell ratio of spleen to tumor cells is 5:1. Fuse a maximum of 1.5-2.5×10⁸ spleen cells per tube. Mix the appropriate volumes of each cell suspension together and centrifuge the cells.
1. In a 37° C. water bath warm the following for each tube of spleen cells:
   small pop top tube containing 1 ml 40% PEG
   small pop top tube containing 1 ml SFM
   50 ml conical tube containing 20 ml SFM
   empty 50 ml conical tube
2. Resuspend the spleen-tumor cell pellet carefully in 0.5 ml of SFM and transfer to a 12 ml round bottom tube. Centrifuge at 700 RPM for 8 minutes to form a loose pellet. Monitor the centrifuge speed and time.
3. Remove all of the supernatant from the pellet.
4. The fusion is done in the 37° C. water bath inside the tissue culture hood.
5. Disrupt the cell pellet by flicking and/or tapping the bottom of the tube.

6. Perform the following steps described below using a stop watch.

| Time (use | |
|---|---|
| stopwatch) | Procedure |
| 00-30 seconds: | Add 0.5 ml PEG; tap the bottom of the tube to mix. |
| 30-60 seconds: | Add remaining 0.5 ml; tap tube to mix. |
| 1-2 minutes: | Over this time period, slowly add 1 ml of SFM while |
| | agitating the tube. |
| 2-6 minutes: | Add 20 ml of SFM over the remaining 4 minutes. As the |
| | volume increases in the original round bottom tube, |
| | transfer the contents into an empty 50 ml conical tube. |

7. Centrifuge the cells and resuspend them in serum-containing medium. For each tube of fused cells, plate the cells into 4-6 96-well plates at 0.1 ml per well (Ten ml of cell solution are needed per plate).
8. Incubate at 37° C.

Day 1

Feed the cells by adding an additional 0.1 ml per well of DMEM with fetal calf serum and HT plus 2× methotrexate. Process and store the antisera.

Day 3

Replace 0.1 ml of media from each well with 0.1 ml of fresh HT media.

Day 7

Repeat the Day 3 procedure.

Day 11

Repeat the Day 3 procedure, and continue to feed every 7 days.

The screening typically occurs between days 11-14.

The supernatants from each well are tested to find those producing the desired anti IFN-γ antibody. Because the original cultures may have been started with more than one hybridoma cell, cells are plated from each antibody-positive culture to isolate pure clones, and subcultured. The sizes of the successful cultures are scaled up. Hybridoma cultures are maintained indefinitely: Antibodies are purified using affinity chromatography with an IFN-γ ligand.

Examples of specific anti-IFN-γ antibodies, and further methods for their preparation and synthesis is described in international patent application WO2004/046306.

Alternatively, but not exclusively, the inhibitor may comprise a soluble protein comprising at least the IFN-γ binding domain of the IFN-γR complex, preferably, for example, the human IFN-γR. The human IFN-γR is a heteromultimeric receptor complex comprising an a subunit (termed IFN-γRα) which is largely responsible for ligand binding and a IFN-γRα subunit (IFN-γRβ) which appears to be essential for cell signaling, apparently by serving to recruit the JAK 1 tyrosine kinase into the receptor complex. Moreover, the association of the IFN-γRα and IFN-γRβ subunits occurs in an IFN-γ-dependent manner. That is, the in situ formation of the complex on the extracellular side of the cell membrane seems to require the presence of IFN-γ.

Thus, as can be seen, an inhibitor of IFN-γ cell signaling activity may be a direct inhibitor (either competitive or non-competitive) of such activity that functions to prevent the binding of IFN-γ to the IFN-γRα subunit. Alternatively, the inhibitor may be an indirect inhibitor of such cell signaling, for example, preventing the association of alpha and beta subunits or of JAK 1 with the Beta subunit, since all these associative events appear to be required in order to raise an effective IFN-gamma cell signaling response.

Since the IFN-γRα subunit is required for interferon binding, and appears to be the IFN acceptor molecule, a soluble version of the alpha receptor (i.e., not imbedded in a cell membrane), for example, one retaining a interferon-binding region but lacking a trans- or intramembrane domain, is a direct inhibitor of IFN-γ activity.

The N terminal portion of the IFN-γRα chain is responsible for IFN binding—this region corresponds approximately to amino acid 1 through amino acid 246 of this polypeptide. This polypeptide can be prepared as follows: Standard PCR is used to amplify the full-length sequence encoding human IFN-γRα from a human lymphoid marathon ready cDNA bank (Clonetech). This sequence is subcloned into an expression plasmid and transfected into CHO cells using the calcium phosphate method. The resputing conditioned media containing the soluble IFN-γRα fragment is concentrated and the receptor fragment purified using Protein G.

In a different embodiment, the inhibitor may comprise a proteinacious or non-proteinacious INF-γ-binding molecule having an association constant to the human IFN-γR substantially similar to, or greater than, that of human IFN-γ. Such a molecule may be easily identified using any of a variety of common compound screening techniques and a library of compounds. The library of compounds may be, for example, peptides or proteins contained in or derived from a phage-display library, a combinatorial library of organic molecules other than macromolecules, and the like.

Chemical libraries are intentionally created collections of different molecules; these molecules can be made by organic synthetic methods or biochemically. In the latter case, the molecules can be made in vitro or in vivo.

Combinatorial chemistry is a synthetic strategy in which the chemical members of the library are made according to a systematic methodology by the assembly of chemical subunits. Each molecule in the library is thus made up of one or more of these subunits. The chemical subunits may include naturally-occurring or modified amino acids, naturally-occurring or modified nucleotides, naturally-occurring or modified saccharides or other molecules, whether organic or inorganic. Typically, each subunit has at least two reactive groups, permitting the stepwise construction of larger molecules by reacting first one then another reactive group of each subunit to build successively more complex and potentially diverse molecules.

By creating synthetic conditions whereby a fixed number of individual building blocks, for example, the twenty naturally-occurring amino acids, are made equally available at each step of the synthesis, a very large array or library of compounds can be assembled after even a few steps of the synthesis reaction. Using amino acids as an example, at the first synthetic step the number of resulting compounds (N) is equal to the number of available building blocks, designated as b. In the case of the naturally-occurring amino acids, b=20. In the second step of the synthesis, assuming that each amino acid has an equal opportunity to form a dipeptide with every other amino acid, the number of possible compounds N=b²=20²=400.

For successive steps of the synthesis, again assuming random, equally efficient assembly of the building blocks to the resulting compounds of the previous step, N=b^{x} where x equals the number of synthetic assembly steps. Thus it can be seen that for random assembly of only a decapeptide the number of different compounds is 20¹⁰ or 1.02×10¹³. Such an extremely large number of different compounds permit the assembly and screening of a large number of diverse candidates for a desired enzymatic, immunological or biological activity.

Biologically synthesized combinatorial libraries have been constructed using techniques of molecular biology in bacteria or bacteriophage particles. For example, U.S. Pat. Nos. 5,270,170 and 5,338,665 to Schatz describe the construction of a recombinant plasmid encoding a fusion protein created through the use of random oligonucleotides inserted into a cloning site of the plasmid. This cloning site is placed within the coding region of a gene encoding a DNA binding protein, such as the lac repressor, so that the specific binding function of the DNA binding protein is not destroyed upon expression of the gene. The plasmid also contains a nucleotide sequence recognized as a binding site by the DNA binding protein. Thus, upon transformation of a suitable bacterial cell and expression of the fusion protein, the protein will bind the plasmid which produced it. The bacterial cells are then lysed and the fusion proteins assayed for a given biological activity. Moreover, each fusion protein remains associated with the nucleic acid which encoded it; thus through nucleic acid amplification and sequencing of the nucleic acid portion of the protein:plasmid complexes which are selected for further characterization, the precise structure of the candidate compound can be determined. The Schatz patents are incorporated herein by reference.

In other biological systems, for example as described in Goedell et al., U.S. Pat. No. 5,223,408, nucleic acid vectors are used wherein a random oligonucleotide is fused to a portion of a gene encoding the transmembrane portion of an integral protein. Upon expression of the fusion protein it is embedded in the outer cell membrane with the random polypeptide portion of the protein facing outward. Thus, in this sort of combinatorial library the compound to be tested is linked to a solid support, i.e., the cell itself. A collection of many different random polypeptides expressed in this way is termed a display library because the cell which produced the protein “displays” the drug on its surface. Since the cell also contains the recombinant vector encoding the random portion of the fusion protein, cells bearing random polypeptides which appear promising in a preliminary screen can be lysed and their vectors extracted for nucleic acid sequencing, deduction of the amino acid sequence of the random portion of the fusion protein, and further study.

Similarly, bacteriophage display libraries have been constructed through cloning random oligonucleotides within a portion of a gene encoding one or more of the phage coat proteins. Upon assembly of the phage particles, the random polypeptides also face outward for screening. As in the previously described system, the phage particles contain the nucleic acid encoding the fusion protein, so that nucleotide sequence information identifying the drug candidate is linked to the drug itself. Such phage expression libraries are described in, for example, Sawyer et al., 4 PROTEIN ENGINEERING 947-53 (1991); Akamatsu et al., 151 J. IMMUNOL. 4651-59 (1993), and Dower et al., U.S. Pat. No. 5,427,908.

While synthesis of combinatorial libraries in living cells has distinct advantages, including the linkage of the compound to be tested with a nucleic acid capable of amplification by the polymerase chain reaction or another nucleic acid amplification method, there are clear disadvantages to using such systems as well. The diversity of a combinatorial library is limited by the number and nature of the building blocks used to construct it; thus modified or R-amino acids or atypical nucleotides may not be able to be used by living cells (or by bacteriophage or virus particles) to synthesize novel peptides and oligonucleotides. There is also a limiting selective process at play in such systems, since compounds having lethal or deleterious activities on the host cell or on bacteriophage infectivity or assembly processes will not be present or may be negatively selected for in the library. Importantly, only peptide or oligonucleotide compounds are made in such systems; thus the diversity of the library is restricted to peptide and polynucleotide macromolecules composed of naturally-occurring monomeric units.

Other approaches to creating molecularly diverse combinatorial libraries employ chemical synthetic methods to make use of atypical or non-biological building blocks in the assembly of the compounds to be tested. Thus, Zuckermann et al., 37 J. MED. CHEM. 2678-85 (1994), describe the construction of a library using a variety of N-(substituted) glycines for the synthesis of peptide-like compounds termed “peptoids”. The substitutions were chosen to provide a series of aromatic substitutions, a series of hydroxylated side substitutions, and a diverse set of substitutions including branched, amino, and heterocyclic structures.

Other workers have used small bi- or multifunctional organic compounds instead of, or in addition to, amino acids for the assembly of libraries or collections compounds of medical or biological interest.

The inhibitors of IFN-γ cell signaling to be used in the methods of the present invention can made using any of these or additional means to generate an agent that inhibits the cell signaling activity of human IFN-γ.

In a preferred embodiment the present invention is based upon the observation that IFN-γ was found to play an initiating role in the initiation and/or development of ocular allergy in the mammalian eye, as measured by infiltration of eosinophils and neutrophils into the conjunctiva. This effect is independent of whether keratoconjunctivitis sicca (KCS) is induced or not. Thus inhibition of one or more cell signaling event in ocular tissue is sufficient to block the role of IFN-γ in the development of allergic conjunctivis. Moreover, despite the fact that allergic conjunctivis has been heretofore thought to be strictly a Th2-mediated event, this result shows that Th1 cells interrelate with Th2 cells in allergic ocular inflammatory diseases.

### DETAILED DESCRIPTION OF THE INVENTION

The present invention is directed to methods for the treatment or prevention of ocular inflammation, preferably allergic ocular inflammation such as allergic conjunctivis, by contacting a mammalian eye with a composition comprising an inhibitor of IFN-γ cell signaling activity (also referred to in this specification as an “IFN-γ inhibitor”). The composition is preferably, although not exclusively, contacted with the eye as a topical agent containing the IFN-γ inhibitor in an ophthalmologically acceptable formulation.

Such a formulation may contain one or more vehicle, solubility enhancing component (SEC), buffer, tonicity agent and stabilizing agent.

Any suitable SEC may be employed in accordance with the present invention. In one embodiment, the SECs include pyrrolinidone components, such as polyvinyl pyrrolidone (povidone), polyvinyl alcohol, and polyoximers. In a preferred embodiment, the SECs include polyanionic components. The useful polyanionic components include, but are not limited to, those materials which are effective in increasing the apparent solubility, preferably water solubility, of poorly soluble IFN-γ inhibitors and/or enhance the stability of the IFN-γ inhibitors and/or reduce unwanted side effects of IFN-γ inhibitors. Furthermore, the polyanionic component is preferably ophthalmically acceptable at the concentrations used. Additionally, the polyanionic component preferably includes three (3) or more anionic (or negative) charges. In the event that the polyanionic component is a polymeric material, it is preferred that each of the repeating units of the polymeric material include a discrete anionic charge. Particularly useful anionic components are those which are water soluble, for example, soluble at the concentrations used in the presently useful liquid aqueous media, such as a liquid aqueous medium containing the IFN-γ inhibitor.

The polyanionic component is preferably sufficiently anionic to interact with the IFN-γ inhibitor. Such interaction is believed to be desirable to solubilize the IFN-γ inhibitor and/or to maintain such IFN-γ inhibitor soluble in the carrier component, for example a liquid medium.

Polyanionic components also include one or more polymeric materials having multiple anionic charges. Examples include:
metal carboxymethylstarchs
metal carboxymethylhydroxyethylstarchs
hydrolyzed polyacrylamides and polyacrylonitriles
heparin
homopolymers and copolymers of one or more of:
   acrylic and methacrylic acids
   metal acrylates and methacrylates
   alginic acid
   metal alginates
   vinylsulfonic acid
   metal vinylsulfonate
   amino acids, such as aspartic acid, glutamic acid and the like
   metal salts of amino acids
   p-styrenesulfonic acid
   metal p-styrenesulfonate
   2-methacryloyloxyethylsulfonic acids
   metal 2-methacryloyloxethylsulfonates
   3-methacryloyloxy-2-hydroxypropylsulonic acids
   metal 3-methacryloyloxy-2-hydroxypropylsulfonates
   2-acrylamido-2-methylpropanesulfonic acids
   metal 2-acrylaamido-2-methylpropanesulfonates
   allylsulfonic acid
   metal allylsulfonate and the like.

In another embodiment, the polyanionic components include anionic polysaccharides which tend to exist in ionized forms at higher pH's, for example, pH's of about 7 or higher. The following are some examples of anionic polysaccharides which may be employed in accordance with this invention.

Polydextrose is a randomly bonded condensation polymer of dextrose which is only partially metabolized by mammals. The polymer can contain a minor amount of bound sorbitol, citric acid, and glucose. Chondroitin sulfate also known as sodium chondroitin sulfate is a mucopolysaccharide found in every part of human tissue, specifically cartilage, bones, tendons, ligaments, and vascular walls. This polysaccharide has been extracted and purified from the cartilage of sharks.

Carrageenan is a linear polysaccharide having repeating galactose units and 3,6 anhydrogalactose units, both of which can be sulfated or nonsulfated, joined by alternating 1-3 and beta 14 glycosidic linkages. Carrageenan is a hydrocolloid which is heat extracted from several species of red seaweed and irish moss.

Maltodextrins are water soluble glucose polymers which are formed by the reaction of starch with an acid and/or enzymes in the presence of water. Other anionic polysaccharides found useful in the present invention are hydrophilic colloidal materials and include the natural gums such as gellan gum, alginate gums, i.e., the ammonium and alkali metal salts of alginic acid and mixtures thereof. In addition, chitosan, which is the common name for deacetylated chitin is useful. Chitin is a natural product comprising poly-(N-acetyl-D-glucosamine). Gellan gum is produced from the fermentation of pseudomonas elodea to yield an extracellular heteropolysaccharide. The alginates and chitosan are available as dry powders from Protan, Inc., Commack, N.Y. Gellan gum is available from the Kelco Division of Merk & Co., Inc., San Diego, Calif. Generally, the alginates can be any of the water-soluble alginates including the alkali metal alginates, such as sodium, potassium, lithium, rubidium and cesium salts of alginic acid, as well as the ammonium salt, and the soluble alginates of an organic base such as mono-, di-, or tri-ethanolamine alginates, aniline alginates, and the like. Generally, about 0.2% to about 1% by weight and, preferably, about 0.5% to about 3.0% by weight of gellan, alginate or chitosan ionic polysaccharides, based upon the total weight of the composition, are used to obtain the gel compositions of the invention.

Preferably, the anionic polysaccharides are cyclized. More preferably, the cyclized anionic polysaccharides include less than ten monomer units. Even more preferably, the cyclized polysaccharides include less than six monomer units.

In one embodiment, a particularly useful group of cyclized anionic polysaccharides includes the cyclodextrins. Examples of the cyclodextrin group include, but are not limited to: α-cyclodextrin, derivatives of α-cyclodextrin, β-cyclodextrin, derivatives of β-cyclodextrin, γ-cyclodextrin, derivatives of γ-cyclodextrin, carboxymethyl-β-cyclodextrin, carboxymethyl-ethyl-β-cyclodextrin, diethyl-β-cyclodextrin, dimethyl-β-cyclodextrin, methyl-β-cyclodextrin, random methyl-β-cyclodextrin, glucosyl-β-cyclodextrin, maltosyl-β-cyclodextrin, hydroxyethyl-β-cyclodextrin, hydroxypropyl-p-cyclodextrin, sulfobutylether-β-cyclodextrin, and the like and mixtures thereof. Sulfobutylether-β-cyclodextrin is a preferred cyclized anionic polyasaccharide in accordance with the present invention. It is advantageous that the SECs, including the above mentioned cyclodextrins, employed in this invention be, at the concentration employed, non-toxic to the mammal, human, to inhibit the present incorporation is administered. As used herein, the term “derivatives” as it relates to a cyclodextrin means any substituted or otherwise modified compound which has the characteristic chemical structure of a cyclodextrin sufficiently to function as a cyclodextrin component, for example, to enhance the solubility and/or stability of active components and/or reduce unwanted side effects of the active components and/or to form inclusive complexes with active components, as described herein.

Although cyclodextrins and/or their derivatives may be employed as SECs, one embodiment of the invention may include SECs other than cyclodextrins and/or their derivatives.

A particularly useful and preferred class of polyanionic component includes anionic cellulose derivatives. Anionic cellulose derivatives include metal carboxymethylcelluloses, metal carboxymethylhydroxyethylcelluloses and hydroxypropylmethylcelluloses and derivatives thereof.

The polyanionic components often can exist in the unionized state, for example, in the solid state, in combination with a companion or counter ion, in particular a plurality of discrete cations equal in number to the number of discrete anionic charges so that the unionized polyanionic component is electrically neutral. For example, the present unionized polyanionic components may be present in the acid form and/or in combination with one or more metals. Since the polyanionic components are preferably ophthalmically acceptable, it is preferred that the metal associated with the unionized polyanionic component be ophthalmically acceptable in the concentrations used. Particularly useful metals include the alkali metals, for example, sodium and potassium, the alkaline earth metals, for example, calcium and magnesium, and mixtures thereof. Sodium is very useful to provide the counter ion in the unionized polyanionic component. Polyanionic components which, in the unionized states, are combined with cations other than H+ and metal cations can be employed in the present invention.

The amount of SEC in the present compositions, if they are present, is not of critical importance. Such amount should be effective to perform the desired function or functions (e.g., increasing solubility, aiding in increasing residence time on the ocular surface, or increasing comfort) in the present composition and/or after administration to the human or animal. In one useful embodiment, the amount of polyanionic component in the present composition is in the range of about 0.1% to about 30% (w/v) or more of the composition. Preferably, the amount of polyanionic component is in the range of about 0.2% (w/v) to about 10% (w/v). More preferably, the amount of polyanionic component is in the range of about 0.2% (w/v) to about 0.6% (w/v). Even more preferably, the polyanionic component is carboxymethylcellulose and is present in the composition in the range of about 0.2% (w/v) to about 0.6% (w/v). A particularly useful concentration of carboxymethylcellulose in the present compositions is about 0.5%.

In one embodiment, the SECs, for example a carboxymethylcellulose, assist in solubilizing the IFN-γ inhibitor(s) in the compositions. In a preferred embodiment, the carboxylmethylcellulose helps solubilize an extracellular portion of the IFN-γR in the compositions.

In one embodiment, the compositions may also include preservative components or components which assist in the preservation of the composition. A preservative may be any pharmaceutically tolerable compound which aid in the prevention of microbial growth in a formulation containing the IFN-γ inhibitor. The preservative components are selected so as to be effective and efficacious as preservatives in the present compositions, that is in the presence of the chosen SEC (if present), such as, for example, the polyanionic component, and preferably have reduced toxicity and more preferably substantially no toxicity when the compositions are administered to a human or animal.

Preferably, the present preservative components or components which assist in the preservation of the composition, preferably the IFN-γ inhibitors therein, are effective in concentrations of less than about 1% (w/v) or about 0.8% (w/v) and may be 500 ppm (w/v) or less, for example, in the range of about 10 ppm (w/v) or less to about 200 ppm (w/v).

Very useful examples of the present preservative components include, but are not limited to oxidative preservative components, for example oxy-chloro components, peroxides, persalts, peracids, and the like, and mixtures thereof. Specific examples of oxy-chloro components useful as preservatives in accordance with the present invention include hypochlorite components, for example hypochlorites; chlorate components, for example chlorates; perchlorate components, for example perchlorates; and chlorite components. Examples of chlorite components include stabilized chlorine dioxide (SCD), metal chlorites, such as alkali metal and alkaline earth metal chlorites, and the like and mixtures therefor. Technical grade (or USP grade) sodium chlorite is a very useful preservative component. The exact chemical composition of many chlorite components, for example, SCD, is not completely understood. The manufacture or production of certain chlorite components is described in McNicholas U.S. Pat. No. 3,278,447, which is incorporated in its entirety herein by reference. Specific examples of useful SCD products include that sold under the trademark Dura Klor by Rio Linda Chemical Company, Inc., and that sold under the trademark Anthium Dioxide by International Dioxide, Inc. An especially useful SCD is a product sold under the trademark Purite® by Allergan, Inc.

Other examples of oxidative preservative components include peroxy components. For example, trace amounts of peroxy components stabilized with a hydrogen peroxide stabilizer, such as diethylene triamine penta(methylene phosphonic acid) or 1-hydroxyethylidene-1,1-diphosphonic acid, may be utilized as a preservative for use in components designed to be used in the ocular environment. Also, virtually any peroxy component may be used so long as it is hydrolyzed in water to produce hydrogen peroxide. Examples of such sources of hydrogen peroxide, which provide an effective resultant amount of hydrogen peroxide, include sodium perborate decahydrate, sodium peroxide and urea peroxide. It has been found that peracetic acid, an organic peroxy compound, may not be stabilized utilizing the present system. See, for example, Martin et al U.S. Pat. No. 5,725,887, the disclosure of which is incorporated in its entirety herein by reference.

Alternatively, or in addition, preservatives other than oxidative preservative components may be included in the compositions. The choice of preservatives may depend on the route of administration. Preservatives suitable for compositions to be administered by one route may possess properties which preclude their administration by another route. Other preferred preservatives may include quaternary ammonium compounds, in particular the mixture of alkyl benzyl dimethyl ammonium compounds and the like known generically as “benzalkonium chloride” or “BAK”. Other quaternary ammonium compounds include Polyquad® (polyquaternium-1), cetrimide (hexadecyltrimethylammonium bromide), and BDB. Among other types of preservatives are the biguanides, such as polyhexamethylene biguanide (PHMB).

Additionally, tonicity adjustors may be added as needed or convenient. They include, but are not limited to, salts, particularly sodium chloride, sodium borate, and potassium chloride, as well as non-salts such as mannitol and glycerin, or any other suitable ophthalmically acceptable tonicity adjustor.

Various buffers and means for adjusting pH may be used so long as the resulting preparation is ophthalmically acceptable. Accordingly, buffers include, but are not limited to, acetate buffers, citrate buffers, phosphate buffers, tris buffers and borate buffers. Acids or bases may be used to adjust the pH of these formulations as needed.

In a similar vein, an ophthalmically acceptable antioxidant that can be used in the present invention includes, but is not limited to sodium metabisulfite, sodium thiosulfate, acetylcysteine, butylated hydroxyanisole, and butylated hydroxytoluene.

Other excipient components which may be included in such ophthalmic preparations are chelating agents which may be added as needed. The preferred chelating agent is ethylene diamine tetraacetic acid (EDTA), although other chelating agents may also be used in place of or in conjunction with it.

In a preferred embodiment of the present invention, the inhibitor of IFN-γ cell signaling activity comprises a protein comprising at least a portion of the extracellular domain of human IFN-γ alpha chain (SEQ ID NO: 1). By protein is meant a peptide, polypeptide or protein. Such a protein will inhibit or lessen binding of IFN-γ to its receptor, and may comprise at least 10 amino acids, or at least 15 amino acids, or at least 20 amino acids, or at least 30 amino acids, or at least 50 amino acids, or at least 70 amino acids, or at least 100 amino acids of the extracellular portion of SEQ ID NO: 1. In a particularly preferred embodiment, the inhibitor comprises amino acid residues 1-146 of the human IFN-γ alpha chain amino acid sequence. The human IFN-γ alpha chain amino acid sequence is provided as SEQ ID NO:1 below:

MALLFLLPLVMQGVSRAEMGTADLGPSSVPTPTNVTIESYNMNPIVYWEYQIMP
QVPVFTVEVKNYGVKNSEWIDACINISHHYCNISDHVGDPSNSLWVRVKARVGQ
KESAYAKSEEFAVCRDGKIGPPKLDIRKEEKQIMIDIFHPSVFVNGDEQEVDYDPE
TTCYIRVYNVYVRMNGSEIQYKILTQKEDDCDEIQCQLAIPVSSLNSQYCVSAEG
VLHVWGVTTEKSKEVCITIFNSSIKGSLWIPVVAALLLFLVLSLVFICFYIKKINPL
KEKSIILPKSLISVVRSATLETKPESKYVSLITSYQPFSLEKEVVCEEPLSPATVPGM
HTEDNPGKVEHTEELSSITEVVTTEENIPDVVPGSHLTPIERESSSPLSSNQSEPGSI
ALNSYHSRNCSESDHSRNGFDTDSSCLESHSSLSDSEFPPNNKGEIKTEGQELITVI
KAPTSFGYDKPHVLVDLLVDDSGKESLIGYRPTEDSKEFS

The ligand binding sequence of IFN-γ comprises the following amino acid sequence (SEQ ID NO: 2):

MKYTSYILAFQLCIVLGSLGCYCQDPYVKEAENLKKYFNAGHSDVADNGTLFLG
ILKNWKEESDRKIMQSQIVSFYFKLFKNFKDDQSIQKSVETIKEDMNVKFFNSNK
KKRDDFEKLTNYSVTDLNVQRKAIHELIQVMAELSPAAKTGKRKRSQMLFRGRR
ASQ

Those of skill in the art are aware how to use recombinant means to construct nucleic acid molecules and by employing genetic engineering techniques widely known in the art could easily construct such a nucleic acid molecule, such as a plasmid or other vector, comprising SEQ ID NO: 2 or an IFN-γ-binding portion thereof. As just one example of such methods, see Sambrook et al., MOLECULAR CLONING: A LABORATORY MANUAL (3d ed. Cold Spring Harbor Laboratory Press 2001), hereby incorporated by reference herein in its entirety. A nucleic acid encoding a portion of SEQ ID NO:2 may encode at least 10 amino acids, or at least 15 amino acids, or at least 20 amino acids, or at least 30 amino acids, or at least 50 amino acids or at least 70 amino acids, or at least 100 amino acids of SEQ ID NO: 2.

The protein ligand binding portion of IFN-γ comprising at least 10 amino acids, or at least 15 amino acids, or at least 20 amino acids, or at least 30 amino acids, or at least 50 amino acids or at least 70 amino acids, or at least 100 amino acids of SEQ ID NO: 2 can be used as a tool for screening compounds able to bind to, and inhibit the receptor-mediated activity of IFN-γ. Moreover, in another embodiment, the ligand-binding portion of IFN-γ (lacking other IFN-γ specific amino acid regions necessary for activity) may itself be used to bind to the IFN-γR as an inhibitor of IFN-γR-mediated cell signaling activity.

While some embodiments of the present invention involve the use of the IFN-γ inhibitors in a formulation for topical administration to the ocular surface, in other embodiments, the invention may involve the expression of the inhibitor of IFN-γ cell signaling activity in vivo. In such embodiments the IFNγ inhibitor comprises a protein encoded by a nucleic acid sequence region comprised in a nucleic acid molecule containing a promoter and other regulatory regions permitting the expression of the protein by the human or other mammal to be treated. Thus, in this embodiment the inhibitor of IFN-γ cell signaling activity is expressed from a nucleic acid vector by the patient and permitted to contact ocular cells over a period of time, thereby providing a therapeutic effect in vivo.

In certain embodiments, the soluble IFNγ inhibitor may be contained in a fusion protein along with a portion of an immunoglobulin in order to prolong the serum half-life of the inhibitor and to increase the avidity of the inhibitor for its ligand. For example, an immunoglobulin Fc region, may advantageously be cloned in frame with the IFN-γ inhibitor, as an IgG1-Fc region does not activate the complement cascade.

A particularly useful therapeutic fusion protein comprises a soluble IFN-γR/Fc fusion, wherein the Fc region is derived from IgG1.

Eukaryotic expression vectors capable of expressing the IFN-γ inhibitor in vivo are known in the art, and include retroviral and adenoviral-derived vectors. Methods of constructing such vectors are also well known, and have been the subject of much work over the last 20 years. However, issues concerning toxicity, replication and recombination, and excessively high levels of transient expression of such vectors have limited their applicability as human therapeutic agents. Thus, while the IFN-γ inhibitor of the present invention may be administered using such viral vectors, the Applicants consider that alternative methods may be preferable.

It has been known for a decade or more that nonviral expression plasmids may be injected into muscle tissue as naked DNA in a saline solution with the result that 1-5% of myocytes may become transfected and are capable of significant expression of reporter genes for periods of up to 19 months; see e.g., Wolff, J. A., Possible Mechanisms Of DNA Uptake In Skeletal Muscle in GENE THERAPEUTICS at 82 (Birkhauser (1995)), which is hereby incorporated by reference herein. This method has been used to successfully deliver DNA encoding a chimeric protein comprising a soluble IFN-γR-Fc chimeric protein to mice for the treatment of lupus and immune-related diabetes; to deliver DNA encoding a chimeric protein comprising complement receptor 1 (CR1)/Fc chimeric protein for the treatment of collagen-induced arthritis; to deliver DNA encoding a chimeric protein comprising a soluble transforming growth factor β1 (TGF-1)/Fc chimeric protein for the treatment of lupus, colitis, streptococcal cell wall (SCW)-induced arthritis, and immune-related diabetes; to deliver DNA encoding a chimeric protein comprising a interleukin 4 (IL-4)/Fc chimeric protein for the treatment of immune-related diabetes; and to deliver DNA encoding a protein comprising interleukin 10 (IL-10) for the treatment of immune-related diabetes. See e.g., Prud'homme, G. J., 22 TRENDS IN IIMMUNOLOGY 149 (Mar. 3, 2001).

Vectors useful for expressing the IFN-γ inhibitor peptide may be any vector capable of being expressed in the host cell or host organism. In the embodiment of the invention in which the plasmid is expressed in vivo, the host organism will be a human or other target mammal. However, the same cloning strategies described above can be used to transform or transfect cells with DNA encoding the IFN-γ inhibitor for expression and purification. In such cases, the IFN-γ inhibitor peptide should be derived from the species to be treated (e.g., human), while the regulatory regions including the promoter, polyadenylation signals (if any), and termination signals should be capable of use in the host organism used for expression. One such plasmid contains the cytomegalovirus (CMV) immediate-early enhancer/promoter, the CMV intron A sequence, a cloning polylinker for insertion of the IFN-γR, and a transcriptional terminator region derived from the rabbit β-globin gene. This plasmid, and its method of construction, are disclosed in Prud'homme, et al., 6 GENE THERAPY 771-777 (1999), hereby incorporated by reference herein.

The following examples are for purposes of illustration only, and are not intended to describe the full scope of the invention, which is defined solely by the claims.

### EXAMPLE 1

In one embodiment of the invention, the extracellular portion of the IFN-γR alpha chain and IgG1 constant heavy-chain cDNA are produced by RT-PCR. RNA extraction, reverse-transcription, and PCR amplification are performed using Pfu DNA polymerase (Stratagene, La Jolla, Calif., USA). The resulting cDNA fragments are designed to generate a full-length IFN-γR/IgG1Fc cDNA segment by PCR. This fragment was then inserted into the EcoRV and EcoRI restriction sites of the VR1255 vector (containing the cytomegalovirus (CMV) immediate-early enhancer/promoter, the CMV intron A sequence, a cloning polylinker for insertion of the IFN-γR, and a transcriptional terminator region derived from the rabbit 13-globin gene), purchased from VICAL. The original luciferase cDNA sequence contained in this vector is deleted. This plasmid directs eukaryotic gene expression. Plasmid DNA is prepared by the alkaline lysis method using an endotoxin-free extraction kit (Qiagen Inc, Santa Clarita, Calif., USA), diluted to 2 μg/μL in sterile saline and stored at −20° C. The supernatants of COS-7 cells transfected with the recombinant IFN-γR/IgG1Fc-containing vector contain a 130-kD fusion protein, which exhibits inhibition of NO release from a macrophage cell line cultured with IFN-γ and lipopolysaccharide.

If the IFN-γ inhibitor plasmid is to be expressed in the organism to be treated it is preferably injected intramuscularly. Optimally, the volume for injection will be between about 25 and about 1000 microliters; however, the specific volume is not critical, and any effective volume may be used. Between about 100 and about 1000 micrograms of the naked plasmid is generally used for injection, although the amount of plasmid may be raised or lowered depending upon the desired dosage and efficiency of transformation and expression of the IFN-γ inhibitor. Injection may be made into the tiballis anterior muscle or alternatively other muscles, such as the rectus femoris or the vastus medialis.

### EXAMPLE 2

A 37 year-old male patient suffering from panuveitis, presenting with symptoms including retinal lesions, vitreal hazing and vasculitis, is injected with an aqueous saline preparation containing 500 micrograms of the plasmid described in Example 1. Within 4 weeks following the date of injection, detectable levels of the IFN-γR/IgG1Fc fusion protein are detected in this patients serum, and remain at such levels for three months without a repeat of the injection. Within 15 days following injection, acute ocular inflammation including retinal lesions and vasculitis, resolved completely. Visual acuity, which is adversely affected by the inflammation, returns to levels which were normal for the patient before his development of panuveitis.

What is claimed is:

1. A method for the treatment of ocular inflammation in a patient comprising administering to said patient a pharmacologically effective dose of an IFN-γ inhibitor.

2. The method of claim 1 in which said IFN-γ inhibitor is selected from the group consisting of a small molecule IFN-γ inhibitor, a polypeptide IFN-γ inhibitor and a nucleic acid which encodes a polypeptide IFN-γ inhibitor when expressed in vivo.

3. The method of claim 2 in which said IFN-γ inhibitor comprises a polypeptide IFN-γ inhibitor.

4. The method of claim 3 in which said polypeptide IFN-γ inhibitor comprises a IFN— binding region contained in an extracellular portion of IFN-γR.

5. The method of claim 4 in which the polypeptide IFN-γ inhibitor comprises a IFN-binding region contained in an extracellular portion of IFN-γR alpha chain.

6. The method of claim 4 in which the polypeptide IFN-γ inhibitor comprises a IFN-binding region contained in an extracellular portion of the human IFN-γR alpha chain.

7. The method of claim 5 in which said extracellular portion of the human IFN-γR alpha chain comprises amino acids 1-146 of SEQ ID NO: 1.

8. The method of claim 2 in which the IFN-γ inhibitor comprises a nucleic acid which encodes a polypeptide IFN-γ inhibitor when expressed in vivo.

9. The method of claim 1 wherein said administration step comprises intramuscular injection.

10. The method of claim 1 wherein said administration step comprises ocular topical administration.

11. A method of treating ocular inflammation in a patient comprising administering to said patient a composition comprising a soluble compound which inhibits IFN-γ-mediated cell signaling in vivo.

12. The method of claim 11 wherein the soluble compound comprises an inactive IFN-γ which will bind to IFN-γR in vivo

13. The method of claim 11 wherein said soluble compound is able to bind at least 10 consecutive amino acids of SEQ ID NO: 2, wherein binding of said soluble compound to a human IGN-γ prevents or lessens IFN-γ binding to the IFN-γR in vivo.

14. The method of claim 13 wherein said soluble compound comprises a monoclonal antibody.

15. The method of claim 13 wherein said soluble compound comprises a ligand binding portion of the IFN-γR.

16. The method of claim 13 wherein said soluble compound comprises a portion of the IFN-γR capable of inhibiting or lessening IFN-γ activity in said patient.

17. The method of claim 16 wherein said soluble compound comprises amino acids 1-146 of SEQ ID NO: 1.
